(19) United States
(12) Patent Application Publication
(10) Pub. No.: US 2010/0278909 A1
(43) Pub. Date: Nov. 4, 2010

(54) PROCESS FOR FORMING SOLID ORAL DOSAGE FORMS OF ANGIOTENSIN II RECEPTOR ANTAGONISTS

(75) Inventors: Ron Schlinger, Tel-Aviv (IL); Avi Avramoff, Haifa (IL)

Correspondence Address: DR. D. GRAESER LTD., 9003 FLORIN WAY, UPPER MARLBORO, MD, 20772, US

(73) Assignee: DEXCEL LTD., Hadera (IL)

(21) Appl. No.: 12/663,288

(22) PCT Filed: May 29, 2008
(86) PCT No.: PCT/IL2008/000732

§ 371 Date: Jul. 20, 2010

Related U.S. Application Data
(60) Provisional application No. 60/924,941, filed on Jun. 6, 2007.

(51) Int. Cl. A61K 31/41; A61K 31/549; A61P 9/12; A61P 13/12; A61P 9/08; A61K 9/28; A61K 9/48

(52) U.S. Cl. 424/452; 514/381; 514/223.5; 424/465

(57) ABSTRACT

A method for producing granules of an angiotensin II receptor antagonist or a pharmaceutically acceptable salt thereof, which comprises: a) mixing the angiotensin II receptor antagonist or pharmaceutically acceptable salt thereof with a melt granulating agent to form a mixture; b) elevating the temperature of the mixture to the melting point of the melt granulating agent to form a solid dispersion of the angiotensin II receptor antagonist in the melt granulating agent; and c) cooling the solid dispersion to form granules; wherein the melt granulating agent is the only granulating agent used to form the granules.

### FIELD OF THE INVENTION

The present invention relates to a novel method of preparation of a formulation comprising an angiotensin II receptor antagonist, and in particular, to a method using melt granulation of solid components.

### BACKGROUND OF THE INVENTION

Angiotensin II is an oligopeptide in the blood that causes vasoconstriction, increased blood pressure, and release of aldosterone from the adrenal cortex. It is derived from the precursor molecule angiotensinogen, a serum globulin produced in the liver. It plays an important role in the renin-angiotensin system.

Angiotensin receptor blockers (ARBs) are drugs that block the action of angiotensin II. As a result, arterial vessels dilate and blood pressure is reduced, thereby making it easier for the heart to pump blood. ARBs can therefore be used to reduce the incidence of heart failure as well as hypertension. In addition, they slow the progression of kidney disease due to high blood pressure or diabetes.

Drugs in this class include candesartan, eprosartan, irbesartan, losartan, olmesartan, telmisartan, valsartan, and pratosartan. The ARBs may be used alone or in combination with other classes of antihypertensive agents that include thiazide diuretics, 13-blockers, calcium channel blockers, rennin inhibitors, and angiotensin-converting enzyme (ACE) inhibitors, both for the treatment of hypertension and congestive heart failure.

Valsartan, a selective ARB, is a well-known antihypertensive agent, which is rapidly absorbed from the gastrointestinal tract after oral administration. The synthesis and use of valsartan are described in U.S. Pat. No. 5,399,578.

EP 914119B1 describes a method of forming a solid oral dosage form of valsartan by slugging using the steps of grinding the active agent and pharmaceutically acceptable additives; compressing the ground mixture within a compaction force range of 25 to 65 kN at a minimum compaction force to form a comprimate; converting the comprimate to form a granulate and compressing the granulate to form the compressed solid oral dosage form. The compaction step is carried out using roller compaction, which produces a comprimate resembling a thin ribbon in segments. The comprimate must then be screened or milled to produce the granulate, which is time consuming and therefore increases the cost of manufacture. Furthermore, the granulate formed has a rather wide particle size distribution and hence highly variable particle sizes.

Melt granulation is one of the most widely applied processing techniques in the array of pharmaceutical manufacturing operations, and may be used for the manufacture of a variety of dosage forms and formulations, such as immediate release and sustained release pellets, granules and tablets. The process has a number of advantages over other commonly used processing techniques. For example, neither solvent nor water is used in the melt granulation process; time-consuming drying steps are eliminated; there are no requirements on the compressibility of active ingredients, and the entire process is simple, continuous and efficient; uniform dispersion of fine particles occurs; good stability is obtained at varying pH and moisture levels; and the resultant products of such a process are safe for use in humans.

In the pharmaceutical industry, the melt granulation technique has been used for various purposes, such as improving the dissolution rate and bioavailability of a drug by forming a solid dispersion or solid solution; controlling or modifying the release of the drug; or masking the bitter taste of an active drug.

Techniques for melt granulation include spray congealing and tumbling melt granulation.

Spray congealing is a melt technique of high versatility. In addition to manufacture of multiparticulate delivery system, it can be applied to process the raw meltable materials into particles of defined size and viscosity values for the melt agglomeration process. Processing of meltable materials by spray congealing involves spraying a hot melt of wax, fatty acid, or glyceride into an air chamber below the melting point of the meltable materials or at cryogenic temperature. Spray-congealed particles (10-3000 μm in diameter) are obtained upon cooling. The congealed particles are strong and nonporous as there is an absence of solvent evaporation. Ideally, the meltable materials should have defined melting points or narrow melting ranges. Viscosity modifier, either meltable or non-meltable at the processing temperature, may be incorporated into the meltable matrix to change the consistency of the molten droplets.

A newer melt agglomeration technique, i.e., tumbling melt granulation, for preparing spherical beads has been reported. A powdered mixture of meltable and non-meltable materials is fed onto the seeds in a fluid-bed granulator. The mixture adheres onto the seeds with the binding forces of a melting solid to form the spherical beads. In preparing the spherical beads, both viscosity and particle size of the meltable materials should be kept at an optimum value. The particle size of a meltable material should be ⅙ or lower than the diameter of the seeds. High-viscosity meltable materials should not be employed to avoid agglomeration of seeds and producing beads of low sphericity.

Both particle size and viscosity of the meltable materials play a significant role in the melt agglomeration process. The control of the melt agglomeration process is best initiated by using meltable materials of controlled properties. For the melt pelletization and melt granulation processes, it is desirable that meltable materials have a high viscosity to improve the mechanical strength of the agglomerates, but a reduced particle size to prevent uncontrollable agglomerate growth. In tumbling melt granulation, small meltable particles with sufficient viscous binding forces are obligatory for the production of spherical beads.

WO 06/113631 discloses compositions comprising an ARB with at least one solubility enhancing agent. The composition of the ARB with the solubility enhancing agents may be prepared by a process of solubilization using melt granulation, in which the solubility enhancing agent is melted. The ARB is then added and mixed with the molten mass, and allowed to solidify to form granules which are then separated from each other. Alternatively, the ARB and the solubility enhancing agent both may be melted together and congealed to room temperature. As no specific teachings are given apart from the above description, it appears that both the solubility enhancing agent and the ARB may be in the molten state during this process.

Polyethylene glycol 6000 is mentioned as one of many examples of a hydrophilic, non-ionic surfactant which may be used as the solubility enhancing agent. However, the description shows that a dispersion of valsartan with PEG 6000 at a ratio of 1:1 has low solubility and effectively teaches away from the use of PEG 6000. The description also does not include a process in which the solubility enhancing agent is the only melt granulating agent.

WO 05/079752 teaches a controlled release pharmaceutical composition having a therapeutically effective amount of one or more pharmacologically active agents having low bioavailability (which may be ARBs); one or more solubilizers; one or more biocompatible swelling agents; and a swelling enhancer to provide retention of the composition in the stomach. Preparation methods may include melt granulation, as described above for WO 06/113631. Again, PEG 6000 is taught as an example of a hydrophilic non-ionic surfactant for use as a solubilizer. Acyclovir is provided as an example of a drug which is solubilized in PEG 6000. A further example of simvastatin is also taught. ARBs are mentioned only as examples of possible active ingredients suitable for use in the composition, but no specific examples are provided.

WO 03/039521 describes a process for the preparation of liquid active ingredients in solid compositions, which comprises adding the liquid active ingredient to a matrix and/or mixture of matrices which are solid at room temperature and liquid at temperatures ranging from 30° C. to 90° C. The matrices are amphiphilic and/or lipophilic compounds. ARBs are mentioned as examples of additional active ingredients which can be added in solid form to the composition.

### SUMMARY OF THE INVENTION

There is thus a widely recognized need for, and it would be highly advantageous to have, a process for preparation of compositions containing ARBs, which are devoid of at least some of the limitations that are known in the art.

The present invention overcomes the limitations of the prior art by providing a simple procedure of forming suitably sized granules without the need for grinding after granulation, starting with active materials and excipients in their solid form.

According to one aspect of the present invention, there is provided a method for producing granules of an angiotensin II receptor antagonist or a pharmaceutically acceptable salt thereof, the method comprising mixing the angiotensin II receptor antagonist or pharmaceutically acceptable salt thereof with a melt granulating agent and optional excipients to form a mixture; elevating the temperature of the mixture to the melting point of the melt granulating agent to form a solid dispersion of angiotensin II receptor antagonist and optional excipients in the melt granulating agent; and cooling the solid dispersion to form granules; wherein the melt granulating agent is the only granulating agent used to form granules.

According to further features of this embodiment of the present invention, the melt granulating agent may optionally consist essentially of PEG 6000.

According to another aspect of the present invention there is provided a method for producing granules comprising excipients, wherein an angiotensin II receptor antagonist or a pharmaceutically acceptable salt thereof is added extra granularly, the method comprising mixing a melt granulating agent and optional excipients to form a mixture; heating the mixture to a temperature greater than the melting point of the melt granulating agent to form a solid dispersion of the excipients in the melt granulating agent; cooling the solid dispersion to form granules; adding the extragranular angiotensin II receptor antagonist or pharmaceutically acceptable salt thereof and optional excipients to the granules.

According to further features in any of the above embodiments of the invention, the granules produced may optionally be compressed to form a tablet. Alternatively, the granules may be filled into a capsule shell.

According to another aspect of the present invention there is provided a composition for oral administration of an angiotensin II receptor antagonist or a pharmaceutically acceptable salt thereof, comprising a melt granulating agent for dispersing the angiotensin II receptor antagonist in a solid dispersion; wherein the composition is in the form of granules and wherein no solubilizing agent is present in an amount sufficient to increase the solubility of the angiotensin II receptor antagonist.

According to further features in any of the embodiments of the invention, the melt granulating agent may be at least one of, Poloxamer, Polyethylene glycol, Acrylic resins, Beeswax, Carnauba wax, Cetyl palmitate, Glyceryl behenate, Glyceryl monostearate, Glyceryl palmtostearate, Glyceryl stearate, Hydrogenated castor oil, Microcrystalline wax, Paraffin wax, Stearic acid, Stearic alcohol and polyethylene glycol-6000, or mixtures thereof. Optionally, the melt granulating agent includes only polyethylene glycol-6000, which is present at a concentration of from about 1 to about 10% total weight of the composition.

According to still further features in any of the embodiments of the invention, the angiotensin II receptor antagonist is at least one of candesartan, eprosartan, irbesartan, losartan, olmesartan, telmisartan, valsartan, and pratosartan, or mixtures thereof.

Optionally and preferably, the angiotensin II receptor antagonist comprises valsartan.

According to yet further features of any of the embodiments of the present invention, the granules may optionally further comprise an additional excipient, such as, for example, at least one of a filler, a binder, a disintegrant, and a lubricant.

According to further features of any of the embodiments of the present invention, the tablet or capsule further comprises an enteric coating.

According to further features of any of the embodiments of the present invention, the method optionally further comprises an additional pharmaceutically active agent, such as, for example, hydrochlorothiazide, which may be present intra-granularly or extra-granularly.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a comparative dissolution study of an exemplary composition prepared in accordance with the principles of the present invention and Diovan®.

### DESCRIPTION OF THE PREFERRED EMBODIMENTS

The present invention is of a simple and efficient method of preparation of a composition for angiotensin II receptor blockers (herein after referred to as “ARB”), including but not limited to valsartan. The method may be carried out in a single receptacle, and produces a high yield.

The method uses melt granulation to prepare a solid dispersion of ARB in a molten melt granulating agent, resulting in a composition which is in the form of suitably sized granules. The method requires an initial grinding only of the melt granulating agent, but not of the ARB. No further grinding step at the end of the process is required. Furthermore, the active ingredient and the optional excipients are mixed in solid form, and remain in solid form throughout the melt granulation process. After the solid dispersion has been prepared, the granulate is then prepared directly from the resultant cooled and solidified material.

Preferably, a single melt granulating agent is used. More preferably, the melt granulating agent is PEG 6000 or others of its functional class, including but not limited to, Poloxamer, Polyethylene glycol, Acrylic resins, Beeswax, Carnauba wax, Cetyl palmitate, Glyceryl behenate, Glyceryl monostearate, Glyceryl palmtostearate, Glyceryl stearate, Hydrogenated castor oil, Microcrystalline wax, Paraffin wax, Stearic acid and Stearic alcohol. Most preferably, the melt granulating agent is used in an amount which is too low to have an effect on solubility, for example from about 1% to about 10% weight per weight of the formulation. Also most preferably, no solubilizing agent is used in the formulation of the present invention or in the process of preparation thereof.

Optionally and preferably, the method is performed in a single receptacle.

The principles and operation of the compositions and methods according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The method comprises melting a melt granulating agent with other components of the composition, such that the components are linked in a solid dispersion by the molten granulating agent. Upon cooling, the granulating agent solidifies and forms granules with the other components. The granules are of a suitable size for either compression, or for filling into capsule shell, such that no additional grinding step is required.

The active ingredient may be mixed with the melt granulating agent and other excipients prior to melting of the melt granulating agent.

Hence, according to a preferred embodiment of the present invention, there is provided a method of producing granules of an angiotensin II receptor antagonist or a pharmaceutically acceptable salt thereof, comprising mixing the angiotensin II receptor antagonist or pharmaceutically acceptable salt thereof with a melt granulating agent, and optional additional ingredients, to form a ground mixture; elevating the temperature of the mixture to the melting point of the melt granulating agent; and cooling the mixture. Granules are formed upon cooling of the mixture.

Alternatively, a mixture of the melt granulating agent and optional excipients may first be heated to above the melting point of the melt granulating agent (but to a temperature less than that of the melting point of the ARB or optional excipients), and cooled to form granules, prior to extra-granular addition of angiotensin II receptor antagonist.

The granules are then either compressed into tablets, or alternatively, filled into capsules.

The present invention further provides a composition for oral administration of an angiotensin II receptor antagonist or a pharmaceutically acceptable salt thereof, comprising a melt granulating agent for dispersing the angiotensin II receptor antagonist in a solid dispersion, wherein the composition is in the form of granules and wherein no solubilizing agent is present in an amount sufficient to increase the solubility of the angiotensin II receptor antagonist.

Any angiotensin receptor blocker is suitable for use in the method or composition of the present invention, such as, for example, candesartan, eprosartan, irbesartan, losartan, olmesartan, telmisartan, valsartan, or pratosartan. Optionally and preferably, the ARB is valsartan.

The granulating agent used is optionally and preferably polyethylene glycol-6000, which is a stable, hydrophilic substance, which can enhance the effectiveness of tablet binders and impart plasticity to granules. The melting point of PEG 6000 is between 55 and 63° C. When used for thermoplastic granulations, a mixture of the powdered constituents with PEG 6000 is heated to 65-70° C. The mass becomes paste-like and forms granules if stirred when cooling. The granulating agent preferably only features PEG 6000.

Optionally and preferably, the method of the present invention is performed in a single receptacle.

Optionally, further excipients may be used in the method or composition of the present invention, such as, for example, a filler, a binder, a disintegrant, a lubricant, a glidant or mixtures thereof.

Examples of suitable fillers include but are not limited to Avicel® (microcrystalline cellulose), lactose, glucose, sucrose, sorbitol, dibasic calcium phosphate, manitol, corn starch, and potato starch.

Optionally and preferably the filler comprises Avicel® PH 102.

Examples of suitable binders include PEG 6000, microcrystalline cellulose, potato starch, wheat starch, corn starch, Povidone (PVP: polyvinyl pyrrolidone), low molecular weight HPC (hydroxypropyl cellulose), HPMC (hydroxypropyl methylcellulose), carboxymethyl cellulose, hydroxyethyl cellulose, ethylcellulose, gelatin, polyethylene oxide, acacia, dextrin, magnesium aluminum silicate, starch, and polymethacrylates, or a mixture thereof.

Optionally and preferably the binder is PEG 6000.

Examples of suitable disintegrants include low-substituted carboxymethyl cellulose sodium, cross-linked polyvinyl pyrolidone, sodium starch glycolate, cross-linked sodium carboxymethyl cellulose, pregelatinized starch, starch, calcium carboxymethyl cellulose, low substituted hydroxypropyl cellulose, magnesium aluminum silicate, alginic acid, sodium alginate, or a mixture thereof.

Optionally and preferably, the disintegrant is sodium starch glycollate.

Examples of suitable lubricants include a stearate of magnesium, aluminum or calcium, talc, sodium stearyl fumarate and glyceryl behenate

Optionally and preferably, the lubricant is magnesium stearate.

The formulation may optionally comprise additional excipients, such as glidant, fillers, surfactants or lubricants.

Examples of suitable glidants include colloidal silica, powdered cellulose, starch, talc and tribasic calcium phosphate.

Examples of suitable surfactants include but are not limited to polysorbate 80 (for example Tween 80), or sodium lauryl sulfate.

One or more of these additives can be selected and used by the skilled artisan having regard to the particular desired properties of the solid oral dosage form by routine experimentation and without undue burden.

The amount of each additive employed, e.g. glidant, binder, disintegrant, filler or diluent and lubricant may vary within ranges conventional in the art. Thus for example the amount of glidant may vary within a range of 0.1 to 10% by weight, the amount of binder may vary within the range of from about 1 to 20% by weight; the amount of disintegrant may vary within a range of from about 2 to 20% by weight; the amount of filler or diluent may vary within a range of from 15 to 70% by weight; whereas the amount of lubricant may vary within a range of from 0.1 to 5% by weight.

The compression of the granules to form tablets may be carried out in a conventional tabletting machine, such as a rotary compression machine, e.g. tablet presses manufactured by Korsch® (Berlin, Germany) and Manesty® (Merseyside, United Kingdom). The tablets may vary in shape, and be, for example, round, oblong, oval, cylindrical, or any other suitable shape, and may vary in size depending on the concentration of the therapeutic agents.

Alternatively the granules can be filled into capsules.

The method of the present invention may optionally further comprise the step of coating the tablet or capsule with an enteric coating, comprising an enteric material such as, for example, hydroxypropyl methylcellulose acetate succinate (hypromellose acetate succinate), cellulose acetate phthalate, hydroxypropyl methyl cellulose phthalate, polyvinyl acetate phthalate, and sodium alginate, Eudragit™; Eudragit L100™; Eudragit L30D™; Eudragit L30D-55 and Eudragit™ or mixtures thereof.

The method of the present invention may optionally further comprises an additional pharmaceutically active agent, such as, for example, hydrochlorothiazide, which may be present either intra-granularly or extra-granularly. Additional aspects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above descriptions, illustrate the invention in a non limiting fashion.

### Example 1

### Valsartan 160 mg tablets

| Intra granular | |
|---|---|
| Valsartan | 57.1% |
| Avicel PH 102 | 29.9% |
| Sodium starch glycollate | 4.0% |
| PEG 6000 | 6.0% |
| Extra granular | |
| Sodium starch glycollate | 2.0% |
| Magnesium stearate | 1.0% |

In this example, valsartan is the active ingredient; Avicel (microcrystalline cellulose) is a filler; sodium starch glycollate is a disintegrant; PEG 6000 is acting as a granulating agent/binder; magnesium stearate is a lubricant.

An optional but preferred method for preparation is described as follows. First, PEG 6000 is ground using a conventional mill, e.g Clit® mill or Apex® mill (Apex, N.Y., USA). The ground PEG 6000 is then mixed with valsartan, sodium starch glycollate and Avicel PH 102 in a V blender with heating capability e.g. that produced by Patterson-Kelley (PR, USA). The mixture is then heated to 70° C. to form a dispersion. The dispersion is cooled to room temperature to form granules. The granules are sieved, preferably using a sieve of pore size 600μ, and then mixed with sodium starch glycollate. Finally magnesium stearate is added and mixed together to form the final blend. The final blend is compressed into tablets.

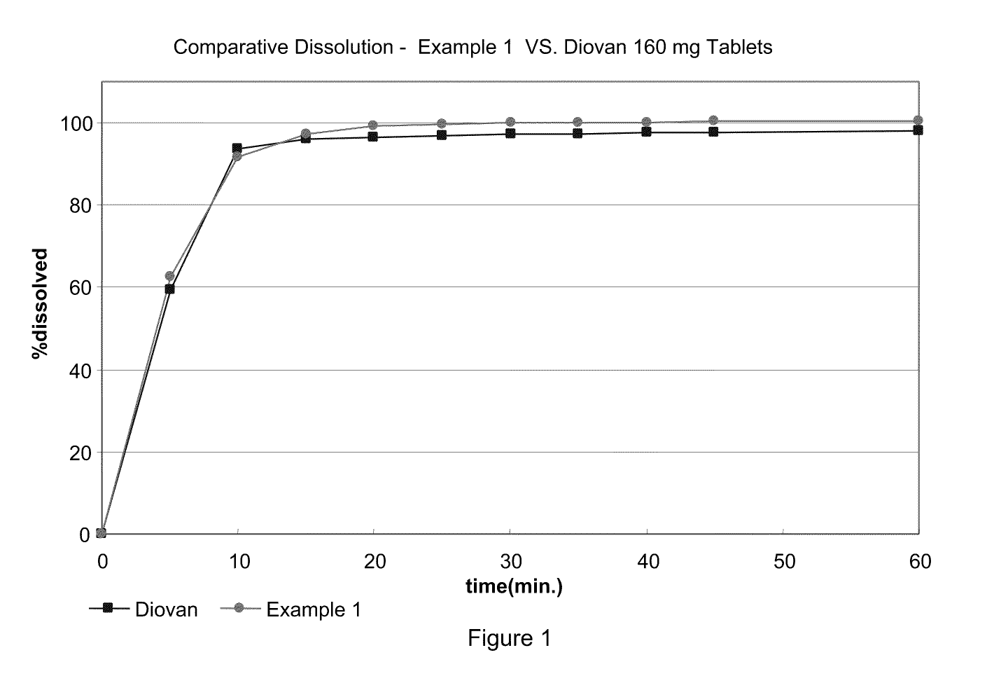

As seen in FIG. 1, a comparative dissolution study of the composition of Example 1 with Diovan® as reference, in phosphate buffer pH 6.8, using apparatus II (paddle) at 50 rpm, showed a substantially identical dissolution profile to that of the reference product.

Alternatively PEG 6000, sodium starch glycollate and Avicel PH 102 are mixed in a V blender with heating capability e.g. that produced by Patterson-Kelley®. The mixture is then heated to 70° C. to form a dispersion. The dispersion is cooled to room temperature to form granules. The granules are sieved (using pore size of 600μ) and then mixed with Valsartan and sodium starch glycollate. Magnesium stearate is then added and mixed to give the final blend which is compressed into tablets.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for producing granules of an angiotensin II receptor antagonist or a pharmaceutically acceptable salt thereof, the method comprising:
a) mixing the angiotensin II receptor antagonist or pharmaceutically acceptable salt thereof with a melt granulating agent and optional excipients to form a mixture;
b) elevating the temperature of said mixture to the melting point of said melt granulating agent to form a solid dispersion of said angiotensin II receptor antagonist and optional excipients in said melt granulating agent; and
c) cooling said solid dispersion to form granules;
wherein said melt granulating agent is the only granulating agent used to form said granules.

2. The method of claim 1, wherein said melt granulating agent consists essentially of PEG 6000.

3. The method of claim 2, performed in a single receptacle.

4. A method for producing granules comprising excipients wherein an angiotensin II receptor antagonist or a pharmaceutically acceptable salt thereof is added extra granularly, the method comprising:
a) mixing a melt granulating agent and optional excipients to form a mixture;
b) heating said mixture to a temperature greater than the melting point of said melt granulating agent to form a solid dispersion of said excipients in said melt granulating agent;
c) cooling said solid dispersion to form granules;
d) adding extra granular angiotensin II receptor antagonist or pharmaceutically acceptable salt thereof and optional excipients to said granules.

5. The method of claim 4, performed in a single receptacle.

6. The method of claim 4, further comprising extra-granular excipients to form a mixture.

7. The method of claim 4, further comprising the step of compressing said granules or mixture to form a tablet.

8. The method of claim 4, further comprising the step of filling said granule, mixture or tablet into a capsule shell.

9. The method of claim 4, wherein said melt granulating agent comprises a melt granulating agent selected from the group consisting of, Poloxamer, Polyethylene glycol, Acrylic resins, Beeswax, Carnauba wax, Cetyl palmitate, Glyceryl behenate, Glyceryl monostearate, Glyceryl palmtostearate, Glyceryl stearate, Hydrogenated castor oil, Microcrystalline wax, Paraffin wax, Stearic acid Stearic alcohol and polyethylene glycol-6000.

10. The method of claim 9, wherein said melt granulating agent includes only PEG 6000 and said polyethylene glycol-6000 is present at a concentration of from about 1 to about 10% total weight of the composition.

11. The method of claim 4, wherein said angiotensin II receptor antagonist is selected from the group consisting of candesartan, eprosartan, irbesartan, losartan, olmesartan, telmisartan, valsartan, and pratosartan.

12. The method of claim 11, wherein said angiotensin II receptor antagonist comprises valsartan.

13. The method of claim 4, wherein said granules further comprise an additional excipient.

14. The method of claim 13, wherein said excipient comprises at least one of a filler, a binder, a disintegrant, and a lubricant.

15. The method of claim 14, wherein said filler is selected from the group consisting of microcrystalline cellulose, lactose, glucose, sucrose, sorbitol, dibasic calcium phosphate, mannitol, corn starch, and potato starch,

16. The method of claim 15, wherein said filler is Avicel® PH 102.

17. The method of claim 14, wherein said binder is selected from the group consisting of polyethylene glycol, microcrystalline cellulose, potato starch, wheat starch, corn starch, Povidone (PVP: polyvinyl pyrrolidone), low molecular weight HPC (hydroxypropyl cellulose), HPMC (hydroxypropyl methylcellulose), carboxymethyl cellulose, hydroxyethyl cellulose, ethylcellulose, gelatin polyethylene oxide, acacia, dextrin, magnesium aluminum silicate, starch, and polymethacrylates, or a mixture thereof.

18. The method of claim 17, wherein said binder includes only polyethylene glycol 6000.

19. The method of claim 14, wherein said disintegrant is selected from the group consisting of low-substituted carboxymethyl cellulose sodium, cross-linked polyvinyl pyrolidone, sodium starch glycolate, cross-linked sodium carboxymethyl cellulose, pregelatinized starch, starch, calcium carboxymethyl cellulose, low substituted hydroxypropyl cellulose, magnesium aluminum silicate, alginic acid, sodium alginate, guar gum, or a mixture thereof.

20. The method of claim 19, wherein said disintegrant comprises sodium starch glycolate.

21. The method of claim 14, wherein said lubricant is selected from the group consisting of a stearate of magnesium, aluminum or calcium, talc, sodium stearyl fumarate or glyceryl behenate.

22. The method of claim 21, wherein said lubricant comprises magnesium stearate.

23. The method of claim 21, further comprising coating said tablet or said capsule further comprises an enteric coating.

24. The method of claim 23, wherein said enteric coating comprises at least one enteric material selected from the group consisting of hydroxypropyl methylcellulose acetate succinate (hypromellose acetate succinate), cellulose acetate phthalate, hydroxypropyl methyl cellulose phthalate, polyvinyl acetate phthalate, alginic acid, and sodium alginate, Eudragit™; Eudragit L100™; Eudragit L30D™; Eudragit L30D-55 and Eudragit™ L or mixtures thereof.

25. The method of claim 4, further comprising an additional pharmaceutically active agent.

26. The method of claim 25, wherein said pharmaceutically active agent is present intra-granularly or extra-granularly.

27. The method of claim 26, wherein said pharmaceutically active agent comprises hydrochlorothiazide.

28. A composition for oral administration of an angiotensin II receptor antagonist or a pharmaceutically acceptable salt thereof, comprising a plurality of granules comprising a solid dispersion of excipients in a melt granulating agent, and said angiotensin II receptor antagonist or a pharmaceutically acceptable salt thereof present only extra granularly; wherein no solubilizing agent is present in an amount sufficient to increase the solubility of the angiotensin II receptor antagonist.

29. The composition of claim 28, wherein said melt granulating agent comprises a melt granulating agent selected from the group consisting of, Poloxamer, Polyethylene glycol, Acrylic resins, Beeswax, Carnauba wax, Cetyl palmitate, Glyceryl behenate, Glyceryl monostearate, Glyceryl palmtostearate, Glyceryl stearate, Hydrogenated castor oil, Microcrystalline wax, Paraffin wax, Stearic acid Stearic alcohol and polyethylene glycol-6000.

30. The composition of claim 29, wherein said melt granulating agent consists essentially of PEG 6000 and said polyethylene glycol-6000 is present at a concentration of from about 1 to about 10% total weight of the composition.

31. The composition of claim 28, wherein said angiotensin II receptor antagonist is selected from the group consisting of candesartan, eprosartan, irbesartan, losartan, olmesartan, telmisartan, valsartan, and pratosartan.

32. The composition of claim 31, wherein said angiotensin II receptor antagonist comprises valsartan.

33. (canceled)

34. The composition of claim 28, wherein said excipient comprises at least one of a filler, a binder, a disintegrant, and a lubricant.

35. The composition of claim 34, wherein said filler is selected from the group consisting of microcrystalline cellulose, lactose, glucose, sucrose, sorbitol, dibasic calcium phosphate, manitol, corn starch, and potato starch,

36. The composition of claim 35, wherein said filler is Avicel® PH 102.

37. The composition of claim 34, wherein said binder is selected from the group consisting of polyethylene glycol, microcrystalline cellulose, potato starch, wheat starch, corn starch, Povidone (PVP: polyvinyl pyrrolidone), low molecular weight HPC (hydroxypropyl cellulose), HPMC (hydroxypropyl methylcellulose), carboxymethyl cellulose, hydroxyethyl cellulose, ethylcellulose, gelatin polyethylene oxide, acacia, dextrin, magnesium aluminum silicate, starch, and polymethacrylates, or a mixture thereof.

38. The composition of claim 37, wherein said binder includes only polyethylene glycol 6000.

39. The composition of claim 34, wherein said disintegrant is selected from the group consisting of low-substituted carboxymethyl cellulose sodium, cross-linked polyvinyl pyrolidone, sodium starch glycolate, cross-linked sodium carboxymethyl cellulose, pregelatinized starch, starch, calcium carboxymethyl cellulose, low substituted hydroxypropyl cellulose, magnesium aluminum silicate, alginic acid, sodium alginate, guar gum, or a mixture thereof.

40. The composition of claim 39, wherein said disintegrant comprises sodium starch glycolate.

41. The composition of claim 34, wherein said lubricant is selected from the group consisting of a stearate of magnesium, aluminum or calcium, talc, sodium stearyl fumarate or glyceryl behenate.

42. The composition of claim 41, wherein said lubricant comprises magnesium stearate.

43. The composition of claim 41, further comprising coating said tablet or said capsule further comprises an enteric coating.

44. The composition of claim 42, wherein said enteric coating comprises at least one enteric material selected from the group consisting of hydroxypropyl methylcellulose acetate succinate (hypromellose acetate succinate), cellulose acetate phthalate, hydroxypropyl methyl cellulose phthalate, polyvinyl acetate phthalate, alginic acid, and sodium alginate, Eudragit™; Eudragit L100™; Eudragit L30D™; Eudragit L30D-55 and Eudragit™ L or mixtures thereof.

45. The composition of claim 28, further comprising an additional pharmaceutically active agent.

46. The composition of claim 45, wherein said additional pharmaceutically active agent is present intra-granularly or extra-granularly.

47. The composition of claim 46, wherein said pharmaceutically active agent comprises hydrochlorothiazide.

48. A method for producing granules comprising excipients wherein an angiotensin II receptor antagonist or a pharmaceutically acceptable salt thereof is added extra granularly, and wherein the angiotensin II receptor antagonist or a pharmaceutically acceptable salt thereof is the sole active ingredient, the method comprising:
a) mixing a melt granulating agent and optional excipients to form a mixture;
b) heating said mixture to a temperature greater than the melting point of said melt granulating agent to form a solid dispersion of said excipients in said melt granulating agent;
c) cooling said solid dispersion to form granules;
d) adding extra granular angiotensin II receptor antagonist or pharmaceutically acceptable salt thereof as the sole active ingredient and optional excipients to said granules.

49. A composition for oral administration of an angiotensin II receptor antagonist or a pharmaceutically acceptable salt thereof as the sole active ingredient, comprising a plurality of granules comprising a solid dispersion of excipients in a melt granulating agent, and said angiotensin II receptor antagonist or a pharmaceutically acceptable salt thereof as the sole active ingredient present only extra granularly; wherein no solubilizing agent is present in an amount sufficient to increase the solubility of the angiotensin II receptor antagonist.
